(19) United States
(12) Patent Application Publication
(10) Pub. No.: US 2008/0299602 A1
(43) Pub. Date: Dec. 4, 2008

(54) Methods of Employing Vascular Leakage to Diagnose a Capillary Leak Disorder

(76) Inventor: Jules B. Puschett, Belton, TX (US)

Correspondence Address: Arnold B. Silverman;Eckert Seamans Cherin & Mellott, LLC, 600 Grant Street, 44th Floor, Pittsburgh, PA, 15219, US

(21) Appl. No.: 12/108,942

(22) Filed: Apr. 24, 2008

Related U.S. Application Data
(60) Provisional application No. 60/932,402, filed on May 31, 2007.
(60) Provisional application No. 61/065,339, filed on Feb. 11, 2008.

(51) Int. Cl. C12Q 1/02; G01N 33/50

(52) U.S. Cl. 435/29; 436/127

(57) ABSTRACT

A method of diagnosing a capillary leak disorder in a patient includes monitoring the marinobufagenin level in blood and/or urine as indicators of vascular permeability and, if a substantial elevation in marinobufagenin exists with respect to that of a normal person, concluding that a capillary leak disorder exists. The method may be employed to diagnose preeclampsia, as well as illnesses or abnormal conditions selected from the group consisting of acute respiratory distress syndrome, hemorrhagic shock, septic, endotoxemia, septicemia, burns.

### CROSS-REFERENCE TO RELATED APPLICATIONS

The present application claims the benefit of U.S. Provisional Patent Application Ser. No. 60/932,402, filed May 31, 2007, and entitled “Method of Diagnosing a Capillary Leak Disorder,” and U.S. Provisional Patent Application Ser. No. 61/065,339, filed Feb. 11, 2008, and entitled “Methods of Employing Vascular Leakage to Diagnose a Capillary Leak Disorder,” the disclosures of which are expressly incorporated herein by reference.

### BACKGROUND OF THE INVENTION

1. Field of the Invention

The present invention relates to methods of employing vascular leakage to diagnose the presence of a capillary leak disorder in a patient to facilitate prompt and effective treatment of the disorder.

2. Description of the Prior Art

While the present invention is not so limited, a primary condition of consequence which may be diagnosed by the methods of the present invention is preeclampsia. Preeclampsia is a condition experienced by pregnant women, and which often involves premature birth. Preeclampsia, if untreated, can progress suddenly to eclampsia, which involves coma and/or convulsive seizures which usually are fatal if untreated.

Preeclampsia is generally characterized by the presence of hypertension, proteinuria, and edema. It is a disorder which generally occurs only in women who are more than twenty weeks pregnant.

Elevated blood pressure or hypertension has long been recognized as a health problem. It is a very common disease which can have widespread effects on a patient's body and frequently, unlike numerous other diseases, is asymptomatic.

Despite means of measuring blood pressure of a patient, as by a sphygmomanometer, for example, there is lacking an accurate, reliable means for detecting the presence of volume dependent hypertension.

U.S. Pat. No. 5,773,076 discloses use of a blood or urine specimen in diagnosing hypertension as indication of acute myocardial infarction. It employs plasma and/or levels of marinobufagenin-like immunoreactivity as a marker for hypertension.

While there is no hard-and-fast rule regarding diagnosis of preeclampsia, several standards have been applied. If a pregnant woman, after twenty weeks of gestation, develops a blood pressure of 140/90 or higher and has edema of the face or hands, and the presence of urinary protein in concentrations greater than 0.3 grams in a twenty-four hour urine collection, this is generally indicative of the presence of preeclampsia.

There remains a very real and substantial need for a method of effectively diagnosing capillary leak disorders.

### SUMMARY OF THE INVENTION

The present invention has met the need for an effective method for diagnosing capillary leak disorders. The method of the invention involves monitoring marinobufagenin in blood and/or urine to determine if a capillary leak disorder exists. If a meaningful increase in marinobufagenin is determined to exist in blood and/or urine, as compared with a normal patient, it is concluded that a capillary leak disorder exists.

A substantial elevation in vascular permeability is deemed to exist when the level of marinobufagenin, as compared with a normal person, is at least about 20 percent greater.

It is an object of the present invention to provide an accurate, efficient, indirect means for rapidly determining if a capillary leak disorder exists in a patient.

It is a further object of the present invention to provide a method for indirect determination of vascular leakage by monitoring marinobufagenin levels in blood and/or urine of animals.

These and other objects of the invention will be more fully understood from the following detailed description of the invention and reference to the illustration appended hereto.

### BRIEF DESCRIPTION OF THE DRAWINGS

FIG. 1 illustrates increasing vascular leakage at time increments from 5 seconds to 50 seconds with Con being the control group.

FIG. 2 is a plot of vascular permeability in three groups of test rats with fluorescence intensity being plotted against time with the upper curve being PDS rats, the intermediate curve being the normal pregnant rats (“NP”), and the lowest curve being the control rats.

### DESCRIPTION OF THE PREFERRED EMBODIMENTS

As employed herein, the term “patient” means members of the animal kingdom, including humans.

As employed herein, the term “capillary leak disorder” means an illness or an abnormal condition which is characterized by vascular leakage of the fluid portion of the blood from the intravascular into the interstitial compartment and shall expressly include, but not be limited to, acute respiratory distress syndrome, hemorrhagic shock, septic endotoxemia, septicemia, burns, endotoxemia, and other illnesses or abnormal conditions characterized by a substantial elevation in marinobufagenin.

As employed herein, “substantial elevation” in vascular permeability means an increase in marinobufagenin with respect to that of a normal person not having a capillary leak disorder of at least about 20 percent.

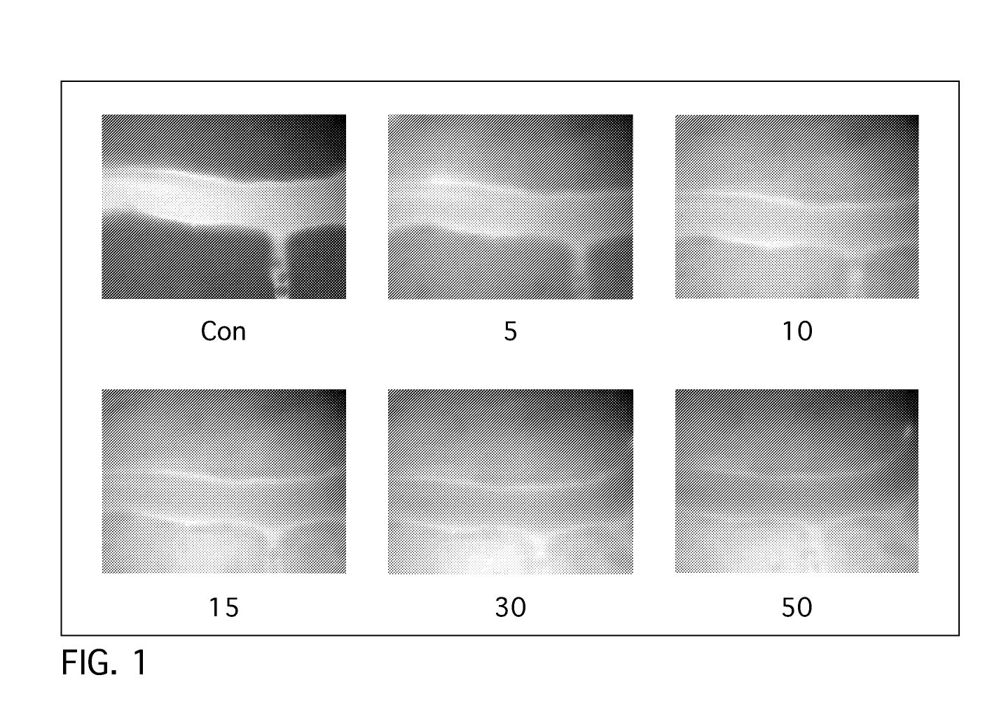

FIG. 1 shows Con being the control and time intervals in minutes related to the extent of leakage. A bolus of 200 nM marinobufagenin was injected intravenously. This shows progressive, large leakage resulting in substantial dye monitored within the extravascular space.

In normal pregnancy, the hematocrit and hemoglobin values fall. This phenomenon is believed to be the result of the expansion of the extracellular fluid volume in which the fluid portion of the blood increases in excess of the increment in the red cell mass. This results in physiologic anemia.

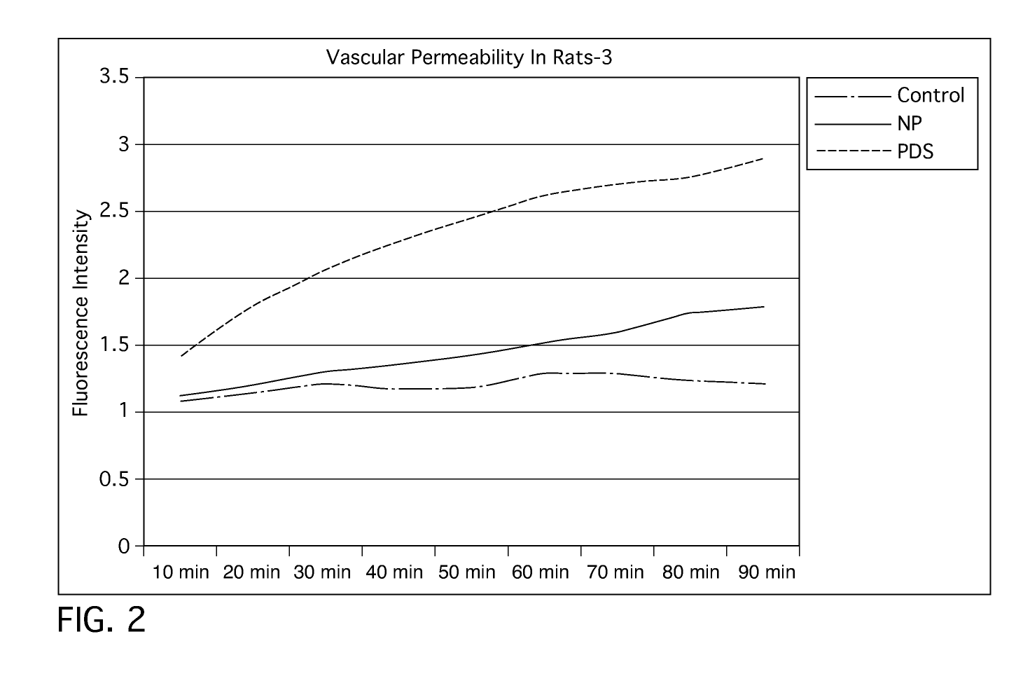

FIG. 2 shows a plot of vascular permeability in rats. The control rats (C) are the nonpregnant rats, the normal pregnant rats (NP), and the animals rendered preeclamptic (PDS) show that the NP rats developed some low-level vascular leakage with the statistical significance becoming evident at 70 minutes. This was deemed consistent with the edema observed in normal pregnant women. The PDS rats developed a significant change in vascular permeability, which became apparent as early as 10 minutes into the procedure.

Considering preeclampsia as an example of capillary leak disorders, patients will have expansion of the extracellular fluid volume with the extra salt and water residing largely in the interstitial and not the intravascular compartment. The fluid portion of the blood, including minerals, such as sodium, potassium, and chloride, for example, and other solutes, such as proteins, will therefore, leak from the plasma in the vascular tree into the interstitium. These alterations can lead to impaired blood flow to the fetus, thereby exacerbating the problem of reduced delivery of nutrients and oxygen to the fetus. This contributes, in many cases, to intrauterine growth retardation.

It has been established that marinobufagenin (“MBG”) is at least a biomarker and perhaps an important pathogenetic factor in the etiology of preeclampsia. The present invention has also established that resibufogenin (“RBG”) is a compound with a similar structure to that of MBG and acts as an antagonist to MBG.

### Example 1

Tests were run on ten rats in the control group, sixteen rats in the normal pregnant group, and fifteen rats in the preeclamptic group (“PDS”). As shown in Table 1, which shows mean values for each group (“NP”), the control group of nonpregnant rats had hematocrit values of 0.51+/−0.02. The second group of rats, which were normal, pregnant rats, had hematocrit values of 0.38+/−0.05. While the preeclamptic rats—designated PDS—had hematocrit values of 0.43+/−0.03, which is substantially higher than the normal pregnant rat. It is also noted that both the normal, pregnant (“NP”) rat hematocrit value and the PDS rats hematocrit value were substantially under the C (control group). In preeclamptic patients, however, the hematocrit rises. It is believed that some of the excess fluid leaks from the intravascular regions into the interstitial compartment. This transudation of the fluid portion at the blood into the space is involved in capillary leak disorders. These results are statistically significantly different from each other.

TABLE 1
Hematocrit Values in Rat Preeclampsia Studies

| | |
|---|---|
| Control (nonpregnant) | 0.51 +/− 0.02 |
| Normal pregnant | 0.38 +/− 0.05 |
| PDS rats | 0.43 +/− 0.03 |

### Example 2

In order to confirm the fact that capillary leak disorders, such as preeclampsia, can be diagnosed by monitoring vascular permeability, a series of tests was performed on rats. Fifteen Sprague-Dawley rats were obtained. Five of the rats were normal, non-pregnant animals and were used as a control group. Five rats were normal pregnant rats (NP). As MBG has a deleterious effect on vascular integrity, and MBG is increased slightly in normal pregnancy and by a great deal in preeclampsia, one would expect an increased vascular leakage in a normal, pregnant rat, as compared with a normal rat, and a further increase in vascular leakage in a preeclamptic rat (“PDS”), as compared with a normal pregnant rat.

In the experimental procedure, single venules were visualized by way of a videomicroscopic technique, which permits viewing blood flow directly. The rats were injected with albumin that had been tagged with fluorescein dye. The distribution of the albumin in the omental vessels of the rat was then observed with a computer being employed to repeatedly measure the dye in the blood vessel and in the interstitium next to the blood vessel, as well as a remote site, which would serve as the control.

A substantial elevation in vascular permeability is deemed to exist if the marinobufagenin in a patient's blood or urine specimen was increased over that of a normal patient by at least about 20 percent.

While for simplicity of disclosure, emphasis has been placed on one of the methods to diagnose preeclampsia, it will be appreciated that it may be employed to diagnose other capillary leak disorders.

It will be appreciated that the present invention provides an efficient, accurate, and simple means for determining whether a patient has a capillary leak disorder.

Whereas particular embodiments of the invention have been described herein for purposes of illustration, it will be evident to those skilled in the art that numerous variations of the details may be made without departing from the invention as set forth in the appended claims.

What is claimed is:

1. A method of diagnosing a capillary leak disorder in a patient comprising:
monitoring the blood and/or urine level of marinobufagenin in said patient, and
if a substantial elevation in marinobufagenin as compared with a normal patient exists, concluding that a capillary leak disorder is present.

2. The method of claim 1 including
employing said method to diagnose the presence of at least one illness or abnormal condition selected from the group consisting of acute respiratory distress syndrome, hemorrhagic shock, septic, endotoxemia, septicemia, and burns.

3. The method of claim 1 including employing said method to diagnose the presence of preeclampsia.

4. The method of claim 2 including said patient is a human being.

5. The method of claim 1 including employing said method on human beings, and
determining that a capillary leak disorder exists if there is an elevation in marinobufagenin level of at least 20 percent as compared with a normal person.

6. The method of claim 1 including monitoring said marinobufagenin in blood.

7. The method of claim 1 monitoring said marinobufagenin in urine.

8. The method of claim 1 monitoring said marinobufagenin in both blood and urine.
